(19) United States
(12) Patent Application Publication
(10) Pub. No.: US 2021/0280282 A1
(43) Pub. Date: Sep. 9, 2021

(54) Anticipatory Analytics Processing of Electronic Medical Records

(71) Applicant: International Business Machines Corporation, Armonk, NY (US)

(72) Inventors: Louis Degenaro, White Plains, NY (US); Edward A. Epstein, Putnam Valley, NY (US); Burn Lewis, Ossining, NY (US); Parthasarathy Suryanarayanan, Croton On Hudson, NY (US)

(21) Appl. No.: 16/811,191

(22) Filed: Mar. 6, 2020

(51) Int. Cl. G16H 10/60; G06Q 10/10

(52) U.S. Cl. CPC G16H 10/60; G06Q 10/1095

(57) ABSTRACT

A mechanism is provided for implementing an anticipatory analytics processing mechanism for processing electronic medical records (EMRs) of a set of patients using anticipatory analytics to provide patient insights. Responsive to receiving a set of EMRs for a set of patients, each EMR in the set of EMRs is queued into one of a plurality of queues based on a set of work types. Responsive to receiving a request for work by a processing service indicating a work type to be provided, an EMR is identified from the plurality of queues based on the work type to be provided and a predetermined order of work. The identified EMR is dispatched to the processing service. Responsive to receiving a completion indication from the processing service, the EMR is either advanced to a queue associated with a next work type category or indicated that patient insight for a patient associated with the EMR is complete.

### BACKGROUND

The present application relates generally to an improved data processing apparatus and method and more specifically to mechanisms for processing electronic medical records of a set of patients using anticipatory analytics to provide patient insights.

Electronic medical records (EMRs) are digital versions of the paper charts in medical professionals' offices, clinics, hospitals, or the like. EMRs contain notes and information collected by and used for the medical professionals in that office, clinic, hospital, or the like, are mostly used by the medical professionals for diagnosis and treatment. EMRs are more valuable than paper records because they enable providers to track data over time, identify patients for preventive visits and screenings, monitor patients, enable computational analyses, and improve health care quality.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method, in a data processing system, comprising at least one processor and at least one memory, where the at least one memory comprises instructions that are executed by the at least one processor to configure the at least one processor to implement anticipatory analytics processing mechanism for processing electronic medical records (EMRs) of a set of patients using anticipatory analytics to provide patient insights. The illustrative embodiment queues each EMR in the set of EMRs into one of a plurality of queues based on a set of work types responsive to receiving a set of EMRs for a set of patients in response to receiving a request for work by a processing service indicating a work type to be provided, identifying an EMR from the plurality of queues based on the work type to be provided and a predetermined order of work. The illustrative embodiment dispatches the identified EMR to the processing service. The illustrative embodiment either advances the EMR to a queue associated with a next work type category or indicates that patient insight for a patient associated with the EMR is complete in response to receiving a completion indication from the processing service.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

The invention, as well as a preferred mode of use and further objectives and advantages thereof, will best be understood by reference to the following detailed description of illustrative embodiments when read in conjunction with the accompanying drawings, wherein:

FIG. 1 is an example diagram of a distributed data processing system in which aspects of the illustrative embodiments may be implemented;

FIG. 2 is an example block diagram of a computing device in which aspects of the illustrative embodiments may be implemented;

FIG. 3 depicts one example of a data processing system that comprises an anticipatory analytics processing mechanism for processing electronic medical records of a set of patients to provide patient insights in accordance with an illustrative embodiment;

FIG. 4 depicts one example of queues that may be utilized by scheduler 304 in accordance with an illustrative embodiment;

FIG. 5 depicts one example of priority windows employed by the dispatcher in accordance with an illustrative embodiment;

FIG. 6 depicts one example of appointment time windows employed by the dispatcher in accordance with an illustrate embodiment;

FIG. 7 depicts one example of a flowchart for the operation performed by a scheduler of an anticipatory analytics processing mechanism in queuing EMRs for processing in accordance with an illustrative embodiment;

FIG. 8 depicts one example of a flowchart for the operation performed by a dispatcher of an anticipatory analytics processing mechanism in queuing EMRs for processing in accordance with an illustrative embodiment; and

FIG. 9 depicts one example of a flowchart for the operation performed by the anticipatory analytics processing mechanism in queuing and dispatching EMRs for processing in accordance with an illustrative embodiment

### DETAILED DESCRIPTION

As stated previously, electronic medical records (EMRs) are digital versions of the paper charts in medical professionals' offices, clinics, hospitals, or the like. EMRs contain notes and information collected by and for the medical professionals in that office, clinic, hospital, or the like, and are mostly used by the medical professionals for diagnosis and treatment. EMRs are more valuable than paper records because they enable providers to track data over time, identify patients for preventive visits and screenings, monitor patients, enable computational analyses, and improve health care quality.

Electronic medical records for patients may be analyzed by intelligent analytics to assist medical professionals with patient insights. Generating patient insights long before patient/medical professional encounters may be sufficient if no changes have occurred in analytics or patient medical records. However, generated patient/medical professional insights may change with the advent of new or updated analytics or with the arrival of new or changed patient data. Therefore, there are advantages in providing the most current insights when patient/medical professional encounters occur.

However, computing resources to produce such patient insights for a plurality of patients are usually not unlimited. That is, it may take many minutes of computing resources/time for analytics to produce patient insights for a single patient and thus, when there are many patients, the computing resources/time for analytics to produce patient insights for all of the patients increase. One simple but expensive approach would be to have on-hand sufficient computing resources to very quickly produce insights for all patients enrolled at a medical practice whenever analytics or patient EMRs change. However, having such on-hand sufficient computing resources comes at a cost to the medical professional.

Therefore, the present invention provides mechanisms that facilitate patient EMR analytics processing in accordance with patient visits while consuming minimal computing resources. When computing resources are limited, the mechanisms process EMRs for a select group of patients, those in close proximity and prior to scheduled interactions with a medical professional. In one embodiment, the mechanisms classify work into a set of categories, such as priorities categories, appointments categories, or the like. Within the priority categories, the mechanisms order or rank EMRs from highest to lowest priority, and, in the case of ties in priority, by order of EMR arrival. Similarly, within appointment categories, the mechanisms order or rank EMRs from an oldest scheduled appointment time (farthest in the past) to a newest scheduled appointment time (farthest in the future), with ties broken by arrival order. The priorities categories may be further classified into a set of subcategories, such as high-priority, low-priority, or the like, and the appointments categories may be further classified into a set of subcategories, such as past appointment, just-missed appointment, imminent appointment, future appointment, or the like. The mechanisms schedule an analysis of corresponding EMRs according to these sets of categories.

In another embodiment, the mechanisms classify work into a set of types, such as notes, summaries, and Delivery. For each EMR, the mechanisms process all sections of the EMR (notes, lab results, diagnoses, histories, immunizations, etc.) resulting in a set of analyzed Notes and results. As an EMR may comprise structured data (e.g. lab results) and unstructured data (e.g. clinician's notes), those data Notes and data Summary utilized by the mechanisms may be implementation specific and may overlap. With the analyzed Notes and obtained results, the processing of the EMR is followed by performing a summarization of the results of this processing, which is then followed by delivering results of the summarization in consumable form as patient insights. The mechanisms dispatch work items with regard to an order or rank of priorities categories, appointments categories, or the like, based on current capabilities of processes on processors based on a type of work the processes can handle at that time. The mechanisms further recalculate the ordering or ranking whenever new EMRs are submitted or when existing EMRs are retracted or modified.

Before beginning the discussion of the various aspects of the illustrative embodiments and the improved computer operations performed by the illustrative embodiments, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on hardware to thereby configure the hardware to implement the specialized functionality of the present invention which the hardware would not otherwise be able to perform, software instructions stored on a medium such that the instructions are readily executable by hardware to thereby specifically configure the hardware to perform the recited functionality and specific computer operations described herein, a procedure or method for executing the functions, or a combination of any of the above.

The present description and claims may make use of the terms “a,” “at least one of,” and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

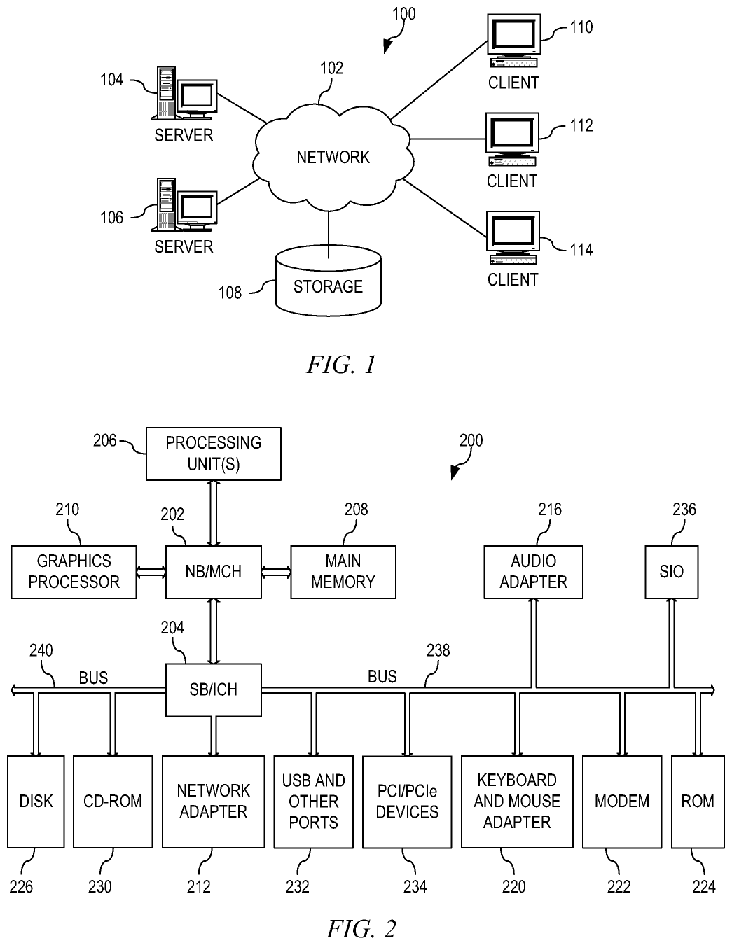

Thus, the illustrative embodiments may be utilized in many different types of data processing environments. In order to provide a context for the description of the specific elements and functionality of the illustrative embodiments, FIGS. 1 and 2 are provided hereafter as example environments in which aspects of the illustrative embodiments may be implemented. It should be appreciated that FIGS. 1 and 2 are only examples and are not intended to assert or imply any limitation with regard to the environments in which aspects or embodiments of the present invention may be implemented. Many modifications to the depicted environments may be made without departing from the spirit and scope of the present invention.

FIG. 1 depicts a pictorial representation of an example distributed data processing system in which aspects of the illustrative embodiments may be implemented. Distributed data processing system 100 may include a network of computers in which aspects of the illustrative embodiments may be implemented. The distributed data processing system 100 contains at least one network 102, which is the medium used to provide communication links between various devices and computers connected together within distributed data processing system 100. The network 102 may include connections, such as wire, wireless communication links, or fiber optic cables.

In the depicted example, server 104 and server 106 are connected to network 102 along with storage unit 108. In addition, clients 110, 112, and 114 are also connected to network 102. These clients 110, 112, and 114 may be, for example, personal computers, network computers, or the like. In the depicted example, server 104 provides data, such as boot files, operating system images, and applications to the clients 110, 112, and 114. Clients 110, 112, and 114 are clients to server 104 in the depicted example. Distributed data processing system 100 may include additional servers, clients, and other devices not shown.

In the depicted example, distributed data processing system 100 is the Internet with network 102 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other computer systems that route data and messages. Of course, the distributed data processing system 100 may also be implemented to include a number of different types of networks, such as for example, an intranet, a local area network (LAN), a wide area network (WAN), or the like. As stated above, FIG. 1 is intended as an example, not as an architectural limitation for different embodiments of the present invention, and therefore, the particular elements shown in FIG. 1 should not be considered limiting with regard to the environments in which the illustrative embodiments of the present invention may be implemented.

As shown in FIG. 1, one or more of the computing devices, e.g., server 104, may be specifically configured to implement an anticipatory analytics processing mechanism for processing electronic medical records of a set of patients to provide patient insights. The configuring of the computing device may comprise the providing of application specific hardware, firmware, or the like to facilitate the performance of the operations and generation of the outputs described herein with regard to the illustrative embodiments. The configuring of the computing device may also, or alternatively, comprise the providing of software applications stored in one or more storage devices and loaded into memory of a computing device, such as server 104, for causing one or more hardware processors of the computing device to execute the software applications that configure the processors to perform the operations and generate the outputs described herein with regard to the illustrative embodiments. Moreover, any combination of application specific hardware, firmware, software applications executed on hardware, or the like, may be used without departing from the spirit and scope of the illustrative embodiments.

It should be appreciated that once the computing device is configured in one of these ways, the computing device becomes a specialized computing device specifically configured to implement the mechanisms of the illustrative embodiments and is not a general purpose computing device. Moreover, as described hereafter, the implementation of the mechanisms of the illustrative embodiments improves the functionality of the computing device and provides a useful and concrete result that facilitates processing electronic medical records of a set of patients using anticipatory analytic processing to provide patient insights.

As noted above, the mechanisms of the illustrative embodiments utilize specifically configured computing devices, or data processing systems, to perform the operations for processing electronic medical records of a set of patients using anticipatory analytic processing to provide patient insights. These computing devices, or data processing systems, may comprise various hardware elements which are specifically configured, either through hardware configuration, software configuration, or a combination of hardware and software configuration, to implement one or more of the systems/subsystems described herein. FIG. 2 is a block diagram of just one example data processing system in which aspects of the illustrative embodiments may be implemented. Data processing system 200 is an example of a computer, such as server 104 in FIG. 1, in which computer usable code or instructions implementing the processes and aspects of the illustrative embodiments of the present invention may be located and/or executed so as to achieve the operation, output, and external effects of the illustrative embodiments as described herein.

In the depicted example, data processing system 200 employs a hub architecture including north bridge and memory controller hub (NB/MCH) 202 and south bridge and input/output (I/O) controller hub (SB/ICH) 204. Processing unit 206, main memory 208, and graphics processor 210 are connected to NB/MCH 202. Graphics processor 210 may be connected to NB/MCH 202 through an accelerated graphics port (AGP).

In the depicted example, local area network (LAN) adapter 212 connects to SB/ICH 204. Audio adapter 216, keyboard and mouse adapter 220, modem 222, read only memory (ROM) 224, hard disk drive (HDD) 226, CD-ROM drive 230, universal serial bus (USB) ports and other communication ports 232, and PCI/PCIe devices 234 connect to SB/ICH 204 through bus 238 and bus 240. PCI/PCIe devices may include, for example, Ethernet adapters, add-in cards, and PC cards for notebook computers. PCI uses a card bus controller, while PCIe does not. ROM 224 may be, for example, a flash basic input/output system (BIOS).

HDD 226 and CD-ROM drive 230 connect to SB/ICH 204 through bus 240. HDD 226 and CD-ROM drive 230 may use, for example, an integrated drive electronics (IDE) or serial advanced technology attachment (SATA) interface. Super I/O (SIO) device 236 may be connected to SB/ICH 204.

An operating system runs on processing unit 206. The operating system coordinates and provides control of various components within the data processing system 200 in FIG. 2. As a client, the operating system may be a commercially available operating system such as Microsoft® Windows®. An object-oriented programming system, such as the Java™ programming system, may run in conjunction with the operating system and provides calls to the operating system from Java™ programs or applications executing on data processing system 200.

As a server, data processing system 200 may be, for example, an IBM eServer™ System P® computer system, Power™ processor based computer system, or the like, running the Advanced Interactive Executive (AIX®) operating system or the LINUX® operating system. Data processing system 200 may be a symmetric multiprocessor (SMP) system including a plurality of processors in processing unit 206. Alternatively, a single processor system may be employed.

Instructions for the operating system, the object-oriented programming system, and applications or programs are located on storage devices, such as HDD 226, and may be loaded into main memory 208 for execution by processing unit 206. The processes for illustrative embodiments of the present invention may be performed by processing unit 206 using computer usable program code, which may be located in a memory such as, for example, main memory 208, ROM 224, or in one or more peripheral devices 226 and 230, for example.

A bus system, such as bus 238 or bus 240 as shown in FIG. 2, may be comprised of one or more buses. Of course, the bus system may be implemented using any type of communication fabric or architecture that provides for a transfer of data between different components or devices attached to the fabric or architecture. A communication unit, such as modem 222 or network adapter 212 of FIG. 2, may include one or more devices used to transmit and receive data. A memory may be, for example, main memory 208, ROM 224, or a cache such as found in NB/MCH 202 in FIG. 2.

As mentioned above, in some illustrative embodiments the mechanisms of the illustrative embodiments may be implemented as application specific hardware, firmware, or the like, application software stored in a storage device, such as HDD 226 and loaded into memory, such as main memory 208, for executed by one or more hardware processors, such as processing unit 206, or the like. As such, the computing device shown in FIG. 2 becomes specifically configured to implement the mechanisms of the illustrative embodiments and specifically configured to perform the operations and generate the outputs described hereafter with regard to the anticipatory analytics processing mechanism that processes electronic medical records of a set of patients to provide patient insights.

Those of ordinary skill in the art will appreciate that the hardware in FIGS. 1 and 2 may vary depending on the implementation. Other internal hardware or peripheral devices, such as flash memory, equivalent non-volatile memory, or optical disk drives and the like, may be used in addition to or in place of the hardware depicted in FIGS. 1 and 2. Also, the processes of the illustrative embodiments may be applied to a multiprocessor data processing system, other than the SMP system mentioned previously, without departing from the spirit and scope of the present invention.

Moreover, the data processing system 200 may take the form of any of a number of different data processing systems including client computing devices, server computing devices, a tablet computer, laptop computer, telephone or other communication device, a personal digital assistant (PDA), or the like. In some illustrative examples, data processing system 200 may be a portable computing device that is configured with flash memory to provide non-volatile memory for storing operating system files and/or user-generated data, for example. Essentially, data processing system 200 may be any known or later developed data processing system without architectural limitation.

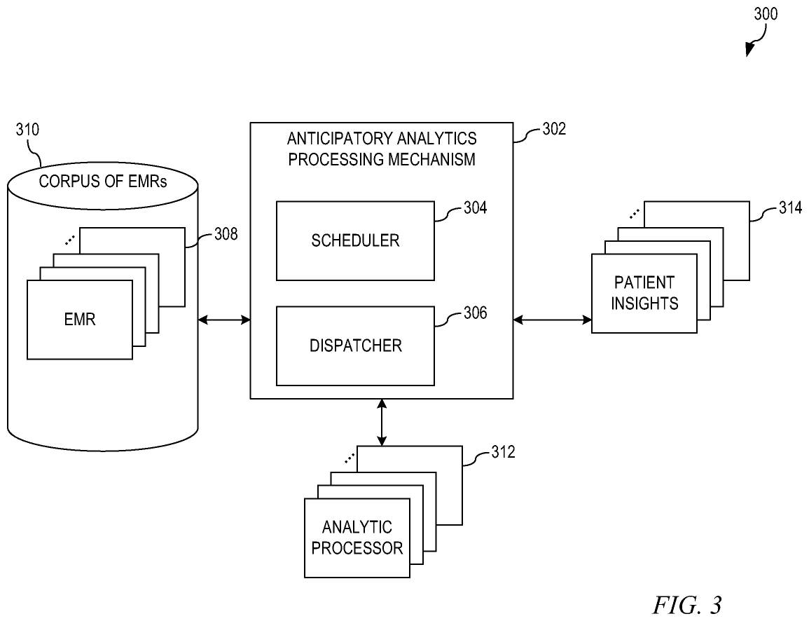

FIG. 3 depicts one example of a data processing system that comprises an anticipatory analytics processing mechanism for processing electronic medical records of a set of patients to provide patient insights in accordance with an illustrative embodiment. Data processing system 300 comprises anticipatory analytics processing mechanism 302 which further comprises scheduler 304 and dispatcher 306. Initially, anticipatory analytics processing mechanism 302 either retrieves current electronic medical records (EMRs) 308 in a corpus of EMRs 310 or identifies EMRs that have been received from, for example from a Fast Healthcare Interoperability Resources (FHIR) server, for a set of patients with scheduled appointments with the medical professional. The EMRs that are retrieved or received may be EMRs that arrived in the corpus of EMRs 310 or directly within anticipatory analytics processing mechanism 302 in batches and sometimes arriving sporadically. However, in either case, the EMRs arrive “prior to” the scheduled appointment, which may be hours, days, weeks, or even months ahead of the scheduled appointment, and are processed by anticipatory analytics processing mechanism 302. In one example, a large EMR (say 500 notes) may arrive 4 weeks ahead of a scheduled visit and may be processed by anticipatory analytics processing mechanism 302. If so, anticipatory analytics processing mechanism 302 saves the results of processing for the date of the patient's appointment. However, if two days before the scheduled appointment, the patient has some lab tests done and a new EMR for the patient is submitted.

Anticipatory analytics processing mechanism 302 utilizes the previous results to the extent possible along with the new lab results to produce revised, up-to-date insights. That is, previously processed Notes results may be reused if the analytics themselves have not changed. Thus, the expectation is that sufficiently prior to a scheduled appointment for each patient for the current day, an up-to-date computation of patient insights is available for the medical professional. However, throughout the current day, unscheduled patients may walk-in for same-day appointments, existing appointments may be canceled or changed, lab tests and other results may become available for previously seen patients as well as patients that are to be seen the current day or patients with future appointments, or the like, all of which affects the desired result of providing up-to-date patient insights for the medical professional in timely fashion. Note also that the deadline for having insights completed is relative to priority and scheduled appointments. With respect to the latter, there may be the cases where some medical care providers may wish to review upcoming scheduled appointments ahead of time, for example 12 or 24 hours beforehand. In such cases, the dispatching windows (discussed below) may be customized accordingly. However, anticipatory analytics processing mechanism 302 is also governed by constraints of limited resource availability and a need for patient insights over a large collection of patient EMRs.

In providing the patient insights, each individual EMR goes through three states of processing: Notes, Summary, and Delivery. This processing order is immutable. That is, all Notes for a particular patient must be analyzed before a Summary is processed and the Summary must be completed before the patient insights are delivered. However, a request for patient insights may enter at any one of the three states, then move forward as the entered state is completed. That is, if the Notes analysis for a particular patient EMR completed but for some reason, such as data processing system 300 encountering an error during processing of at a particular stage, data processing system 300 shutting down for some unexpected reason, maintenance, or the like, then, when data processing system 300 recovers, an EMR for which Notes analysis has completed may reenter the processing at the Summary stage. Likewise, a Summary that completed but that was not delivered may reenter at the Delivery stage.

Thus, as anticipatory analytics processing mechanism 302 retrieves current electronic medical records (EMRs) 308 for a set of patients with scheduled appointments with the medical professional (appointment patients) as well as those unscheduled patients may walk-in for same-day appointments and those previously seen patients for which lab tests and other results become available (priority patients), scheduler 304 organizes, i.e. queues, the submissions in an optimized order to meet the objective of completing up-to-date insights processing in time for medical professional/patient visits or call backs. For each EMR that is retrieved, scheduler 304 determines whether the EMR has a denoted priority indicator. The priority indicator may be an indicator that identifies how important that EMR is, such as a priority indicator if 10 indicating a high importance and a priority indicator of 1 indicating a low importance, as one example. The illustrative embodiments recognize that any type of priority may be used without departing from the spirit and scope of the invention. Therefore, if the EMR has a priority indicator, scheduler 304 determines whether the EMR has a Notes state, i.e. if the EMR has not been analyzed to create a set of processed Notes analyses and results. If all of the EMR Notes have not been analyzed, scheduler 304 queues the EMR into a priority Notes queue. If all the EMR Notes have been analyzed, scheduler 304 determines whether the EMR has satisfied a Summary state, i.e. if the notes for the EMR have been summarized. If the EMR has not been summarized, scheduler 304 queues the EMR into a priority Summary queue. If the EMR has been summarized, then scheduler 304 queues the EMR into a priority Delivery queue.

A similar operation is performed for EMRs that do not have a priority indicator, e.g. but instead have an appointment indicator. That is, if the EMR fails to have a priority indicator, scheduler 304 determines whether the EMR has a Notes state, i.e. if the EMR has not been analyzed to create a set of processed Notes analyses and results. If all of the EMR Notes have not been analyzed, scheduler 304 queues the EMR into an appointment Notes queue. If all of the EMR Notes have been analyzed, scheduler 304 determines whether the EMR has satisfied a Summary state, i.e. if the notes for the EMR have been summarized. If the EMR has not been summarized, scheduler 304 queues the EMR into an appointment Summary queue. If the EMR has been summarized, then scheduler 304 queues the EMR into an appointment Delivery queue.

Within each of the priority Notes queue, the priority Summary queue, and the priority Delivery queue, the EMRs are ordered or ranked based on priority from highest priority to lowest priority. Dispatcher 306 utilizes this ordering or ranking when dispatching EMRs, which will be described hereafter. Within each of the appointment Notes queue, the appointment Summary queue, and the appointment Delivery queue, the EMRs may be ordered or ranked based on appointment time from oldest past appointment to farthest future appointment. This ordering or ranking may be further segmented into “windows of time” such that, for example, EMRs that are older than a first predetermined time are considered past appointments, EMRs that are not older than the first predetermined time up to a second predetermined time are considered just missed appointments, appointments that are from the second predetermined time to a third predetermined time are considered imminent appointments, and appointments that extend beyond the third predetermined time are considered future appointments.

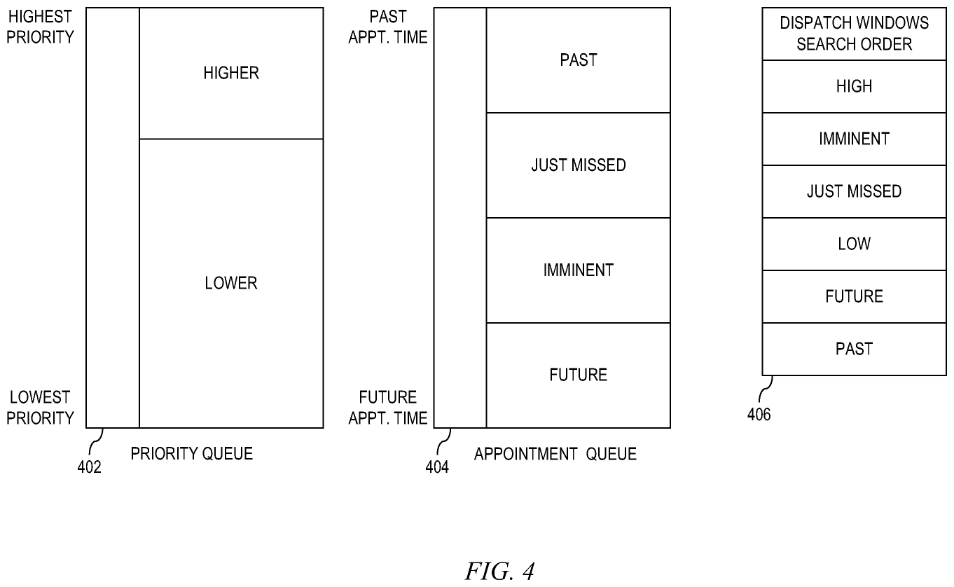

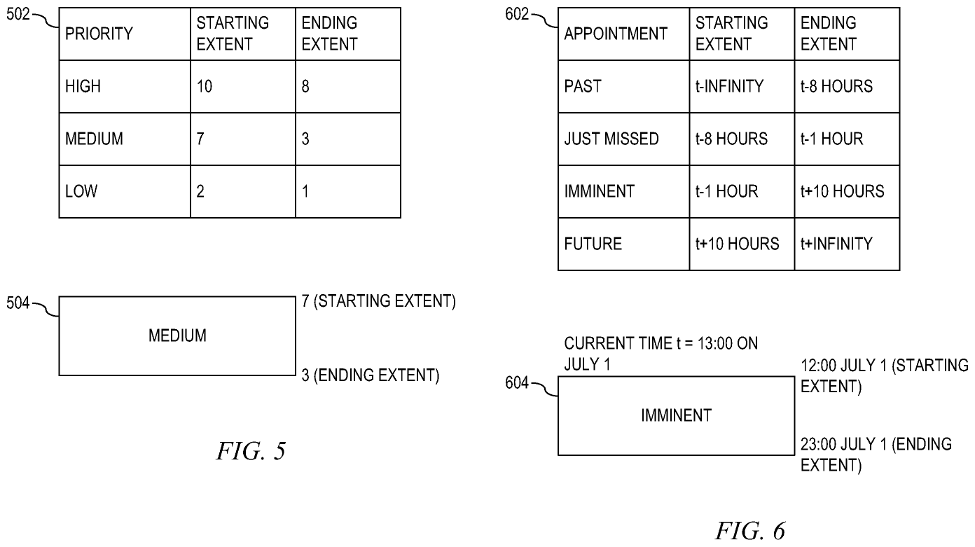

FIG. 4 depicts one example of queues that may be utilized by scheduler 304 and windows employed by the dispatcher 306 in accordance with an illustrative embodiment. As stated above, for each of the Notes, Summary, and Delivery stages, scheduler uses a priority queue 402 and an appointment queue 404. Scheduler 304 orders or ranks the EMRs placed in priority queue 402 the based on priority from highest priority to lowest priority. Within priority queue 402, the high-priority window may have, for example, a starting extent of 10 and an ending extent of 8, while the low-priority window may have, for example, a starting extent of 7 and an ending extent of 1. FIG. 5 depicts one example of priority windows employed by the dispatcher 306 in accordance with an illustrative embodiment. Table 502 illustrates the extents for three priority windows: a high-priority window, a medium-priority window, and a low-priority window. In this example, the high-priority window has a starting extent of 10 and an ending extent of 8, the medium-priority window has a starting extent of 7 and an ending extent of 7, and the low-priority window has a starting extent of 2 and an ending extent of 1. Window 504 illustrates one example priority window, i.e. a medium priority window, with starting extent 7 and ending extent 3, in correspondence with priority queue 402.

Further, for each of the Notes, Summary, and Delivery stages, scheduler 304 orders or ranks EMRs placed in appointment queue 404 based on appointment time from oldest past appointment to farthest future appointment in the appointment queue. This ordering or ranking may be further segmented into dynamically calculated windows such that, for example, EMRs considered past appointments may have, for example, a starting extent of t−infinity and a ending extent of t−8 hours, EMRs considered just missed appointments may have, for example, a starting extent of t−8 hours and a ending extent of t−1 hour, EMRs considered imminent appointments may have, for example, a starting extent of t−1 hour and a ending extent of t+10 hours, and appointments considered future appointments may have, for example, a starting extent of t+10 hours and an ending extent of t+infinity. FIG. 6 depicts one example of appointment time windows employed by the dispatcher 306 in accordance with an illustrate embodiment. Table 602 illustrates the extents for four appointment windows: a past window, a just-missed window, an imminent window, and future window with their configured starting and ending extents. Window 604 illustrates one example appointment time window, i.e. an imminent appointment window, with starting extent 12:00 on July 1 and ending extent 23:00 on July 1 for a current time of 13:00 on July 1, in correspondence with the appointment queue 404.

The “windows” shown in FIG. 4 are defined over each of the queues. Thus, these nominally non-overlapping windows have bounds (or extents), for example a starting time and ending time which are calculated dynamically based on the current time-of-day when an analytics processor request for work is being serviced by the dispatcher. One skilled in the art may easily contemplate an expanded or contracted set of windows over each queue, as well as an expanded or contracted size of each window. Therefore, the extents for the windows in the priority queue are beginning and ending priority, while the extents for the windows in the appointments queue are beginning and ending time. As is also illustrated in FIG. 4, the windows of priority queue 402 and appointment queue 404 may be configured in a dispatch windows search order employed by dispatcher 306, described hereafter. Table 406 illustrates one example of a configured order, from top to bottom of table 406, employed when the dispatcher searches for work when requested by an analytics processor 312, in correspondence with the current time at the time of a request with respect to the configured appointment time windows 602 of FIG. 6 and priorities with respect to the priority windows 502 of FIG. 5.

Returning to FIG. 3, it should be noted that, while the example above indicates separate priority Notes queue, priority Summary queue, and priority Delivery queue as well as separate the appointment Notes queue, appointment Summary queue, and appointment Delivery queue, in one embodiment the separate priority Note queue and priority Summary queue may be collapsed into one combined queue. Similarly, the separate appointment Notes queue and appointment Summary queue may be collapsed into one combined queue. Other similar alternatives and combinations may easily be contemplated by those skilled in the art. Combining queues is useful when scaled-out processing services are capable of processing more than one type of work, i.e. either a Note or a Summary. This type of configuration has the advantage of giving better service utilization during times when one type of work (Note or Service) queue is empty while the other is not.

It should also be noted that the scheduler 304 queues those EMRs for patients continuously. EMRs to be scheduled may arrive for scheduling in batches, or when event occurs such as a new, changed or canceled patient appointment or new or updated lab results.

While scheduler 304 is queuing EMRs, anticipatory analytics processing mechanism 302 may also receive requests from one or more analytics processors in a collection of analytics processors 312 indicting that the one or more analytics processors 312 are available for work. While the term analytics processor is used in this description, it should be noted that this is just one example and analytics processors may be processes on a same or different processor, threads on a same or different processor, or the like. The request from each analytics processor 312 may indicate a type of work that analytics processor 312 may be available to perform, such as Notes work, Summary work, Delivery work, or any combination thereof. Upon receiving the requests and based on the type of work the particular analytics processor 312 has indicated, dispatcher 306 searches for associated work in queues accordingly. In searching for work, dispatcher 306 may follow a predetermined order of work scheduling, for example: high-priority EMRs, imminent appointment EMRs, just missed appointment EMRs, low-priority EMRs, future appointment EMRs, and then past appointment EMRs. It should be noted that high-priority EMRs are differentiated from low priority EMRs based on a priority threshold that may be set and adjust by the user. In should be noted that while the instant example illustrates only two levels of priority—high and low, the illustrative embodiments are not limited to only two levels. That is any number of levels of priority may be used, for example 3 priorities—low, medium, and high, with a revised dispatching scheme accordingly.

Therefore, for the type of work that a particular analytics processor 312 is able to perform, dispatcher 306 searches the associated priority queue/appointment queue using the predetermined order of work until either a unit of work is found or all queue windows have been visited, which ever happens first. In the illustrative embodiments, a “unit of work” may be a bundle of notes, a summarization, or a delivery. More specifically with regard to the predetermined order of work, dispatcher 306 initially searches the priority queue for a unit of work that is within the high-priority window, e.g. within the high-priority window extents. If present, dispatcher 306 assigns the unit of work with the highest priority within that high-priority window to the particular analytics processor 312. If no unit of work within the high-priority window is present in the priority queue, dispatcher 306 searches the appointment queue for a unit of work considered imminent, e.g. within the imminent window extents. If one or more units of work are considered imminent, dispatcher 306 selects the unit of work considered imminent closest to the starting extent of the imminent window and assigns the unit of work to the particular analytics processor 312.

If no unit of work considered imminent is present in the appointment queue, dispatcher 306 searches the appointment queue for a just missed unit of work, e.g. within the just missed window extents. If one or more units of work are considered just missed, dispatcher 306 selects the unit of work considered just missed closest to the starting extent of the just missed window and assigns the unit of work to the particular analytics processor 312. If no unit of work considered just missed is present in the appointment queue, dispatcher 306 searches the priority queue for a unit of work that is within the low-priority window, e.g. within the low-priority window extents. If present, dispatcher 306 assigns the unit of work with the highest priority within that low-priority window to the particular analytics processor 312. If no unit of work within the low-priority window is present in the priority queue, dispatcher 306 searches the appointment queue for a unit of work considered future, e.g. within the future window extents. If one or more units of work are considered future, dispatcher 306 selects the unit of work closest to the starting extent of the future window and assigns the unit of work to the particular analytics processor 312.

If no unit of work considered future is present in the appointment queue, dispatcher 306 searches the appointment queue for a unit of work considered past, e.g. within the past extents. If one or more units of work are considered past, dispatcher selects the unit of work closest to the starting extent of the past window and assigns the unit of work to the particular analytics processor 312. If no unit of work considered past is present in the appointment queue, dispatcher 306 returns a signal that no unit of work is available to the analytics processor 312. At the beginning of each search, the dispatcher 306 dynamically calculates queue search window extents for past appointments, just-missed appointments, imminent appointments, or future appointments based on the current time-of-day with respect to a request for work from a processing service to facilitate selection of an EMR.

In accordance with the illustrative embodiments, any unit of work that dispatcher 306 dispatches to an analytics processor 312 and for which a response is received, scheduler 304 moves the EMR to the next queue. For example, once an analytics processor 312 completes a Notes analysis of all Notes in the EMR, the analytics processor 312 notifies anticipatory analytics processing mechanism 302, which causes scheduler 304 to queue the EMR in the Summary queue. In the illustrative embodiment, different portions of Notes for a particular EMR may be processed by two or more processors. Thus, several processors may be processing disjoint bundles of Notes for the same EMR and anticipatory analytics processing mechanism 302 will wait for all Notes processing for a particular EMR to complete before changing the state of the EMR from Notes to Summary. Similarly, once an analytics processor 312 completes a Summary analysis, the analytics processor 312 notifies anticipatory analytics processing mechanism 302, which causes scheduler 304 to queue the EMR in the Delivery queue. Finally, once an analytics processor 312 completes a Delivery of patient insights 314, the analytics processor 312 notifies anticipatory analytics processing mechanism 302, which causes anticipatory analytics processing mechanism 302 to show the patient insights 314 for the EMR complete.

In accordance with the illustrative embodiments, any unit of work that dispatcher 306 dispatches to an analytics processor 312 and for which no response is received, i.e. no analyzed notes are received, no summary is received, or no confirmation of summary delivery is received, which may occur for a variety of reasons such as, processing service crashing, communication issues failing, infrastructure failures, or the like, before the analytics processor 312 completed the unit of work, dispatcher 306 may detect this situation and recover by requesting that scheduler 304 retry the unit of work. Thus, scheduler 304 re-queues the unit of work for subsequent re-dispatching.

In another embodiment, if an updated EMR is loaded into the corpus of EMRs 310, such as receiving new data lab results, diagnoses, histories, immunizations, or the like, scheduler 304 compares the newly submitted EMR against EMRs already queued. If the newly submitted EMR is not found in the set of queued EMRs, the scheduler 304 adds the EMR to the queue. If the EMR is already queued, scheduler 304 replaces the already queued EMR if possible with additional structured/unstructured data to be analyzed. Further, if a priority or an appointment time associated with an EMR changes, scheduler 304 may move the ERM's place in the associated queue or move the EMR to a different queue. However, if the EMR cannot be replaced because it is currently being processed, then scheduler 304 either rejects the submit request or holds the EMR pending completion of the active work for automatic re-submission.

In yet another embodiment and so as to not waste processing time by the set of analytics processors 312, if a scheduled patient cancels an appointment and the EMR is already queued, scheduler 304 may remove the EMR from the queue. It should also be noted that scheduler 302 provides a query interface that returns information comprising the current queued status of EMRs, including but not limited to the current queues on which an EMR resides and its order in the queue. Further, dispatcher 306 provides a query interface that returns information comprising the current dispatched work items, including but not limited to an EMR work item unique identifier and a dispatched-to service processor location. The returned query results may be useful to, for example, administrators and monitoring programs to determine the current status of EMRs with respect to scheduling, dispatching and processing mechanisms and for assessing the health of same with respect to producing patient insights.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

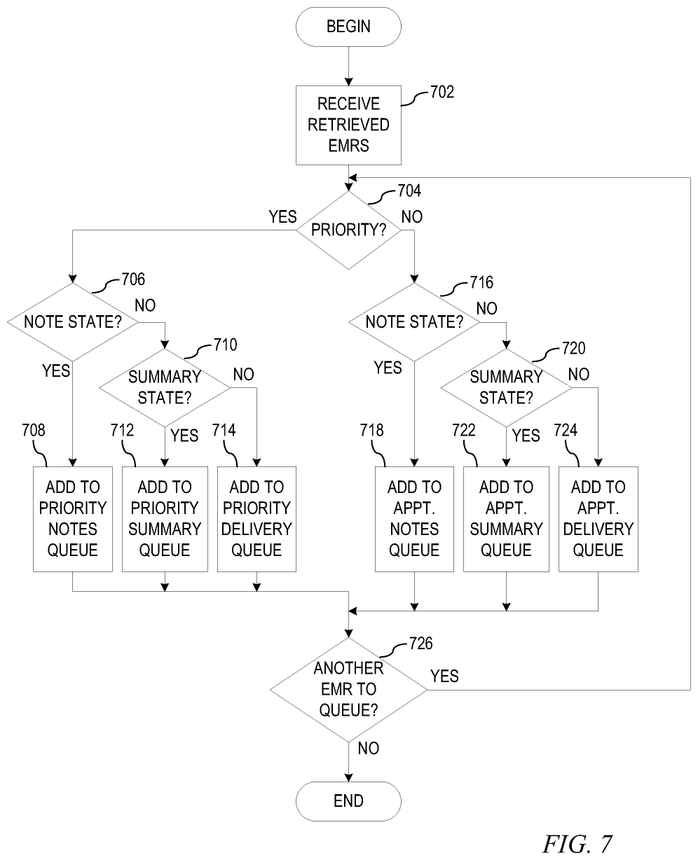

FIG. 7 depicts one example of a flowchart for the operation performed by a scheduler of an anticipatory analytics processing mechanism in queuing EMRs for processing in accordance with an illustrative embodiment. As the operation begins, the scheduler receives a retrieved set of electronic medical records (EMRs) for a set of patients with scheduled appointments with the medical professional (appointment patients) as well as those unscheduled patients that may walk-in for same-day appointments and those previously seen patients for which lab tests and other results become available (priority patients) (step 702). For each EMR that is retrieved, the scheduler determines whether the EMR has a denoted priority indicator (step 704). The priority indicator may be an indicator that identifies how important that EMR is, such as a priority indicator if 10 indicating a high importance and a priority indicator of 1 indicating a low importance, as one example. The illustrative embodiments recognize that any type of priority may be used without departing from the spirit and scope of the invention. If at step 704 the EMR has a priority indicator, the scheduler determines whether the EMR has a Notes state, i.e. if the EMR has not been analyzed to create a set of processed Notes analyses and results (step 706). If at step 706 all of the EMR Notes have not been analyzed, the scheduler queues the EMR into a priority Notes queue (step 708). If at step 706 all the EMR Notes have been analyzed, the scheduler determines whether the EMR has satisfied a Summary state, i.e. if the notes for the EMR have been summarized (step 710). If at step 710 the EMR has not been summarized, the scheduler queues the EMR into a priority Summary queue (step 712). If at step 710 the EMR has been summarized, then the scheduler queues the EMR into a priority Delivery queue (step 714).

If at step 704 the EMR fails to have a priority indicator, scheduler determines whether the EMR has a Notes state, i.e. if the EMR has not been analyzed to create a set of processed Notes analyses and results (step 716). If at step 716 all of the EMR Notes have not been analyzed, the scheduler queues the EMR into an appointment Notes queue (step 718). If at step 716 all of the EMR Notes have been analyzed, the scheduler determines whether the EMR has satisfied a Summary state, i.e. if the notes for the EMR have been summarized (step 720). If at step 720 the EMR has not been summarized, scheduler queues the EMR into an appointment Summary queue (step 722). If at step 722 the EMR has been summarized, then the scheduler queues the EMR into an appointment Delivery queue (step 724). From steps 708, 712, 714, 718, 722, and 724, the scheduler determines whether there is another EMR to queue (step 726). If at step 726 there is another EMR to queue, then the operation returns to step 704. If at step 726 there is not another EMR to queue, then the operation terminates.

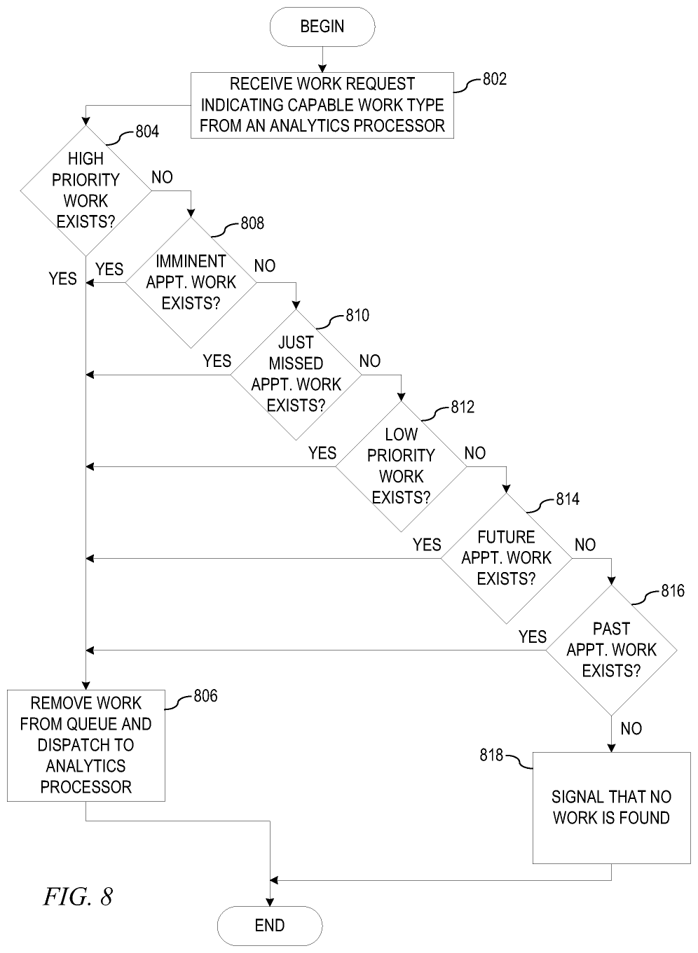

FIG. 8 depicts one example of a flowchart for the operation performed by a dispatcher of an anticipatory analytics processing mechanism in queuing EMRs for processing in accordance with an illustrative embodiment. As the operation begins, the dispatcher receives a request from an analytics processor indicting that the analytics processor is available for work and the type of work that the analytics processor can perform (step 802). Thus, for the type of work that the analytics processor is able to perform, the dispatcher searches the priority queue for a unit of work that is within the high-priority window, e.g. within the high-priority window extents (step 804). If at step 804 a high-priority unit of work is present, then the dispatcher removed the unit of work from the queue and dispatches the unit of work to the analytics processor (step 806), with the operation terminating thereafter. If at step 804 no unit of work with a priority equal to or above the priority threshold is present in the priority queue, the dispatcher searches the appointment queue for a unit of work considered imminent, e.g. within the imminent window extents (step 808). If at step 808 one or more units of work are considered imminent, the dispatcher selects the unit of work considered imminent closest to the starting extent of the imminent window and the operation proceeds to step 806.

If at step 808 no unit of work considered imminent is present in the appointment queue, the dispatcher searches the appointment queue for a just missed unit of work, e.g. within the just missed window extents (step 810). If one or more units of work are considered just missed, the dispatcher selects the unit of work considered just missed closest to the starting extent of the just missed window and the operation proceeds to step 806. If at step 810 no unit of work considered just missed is present in the appointment queue, the dispatcher searches the priority queue for a unit of work that is within the low-priority window, e.g. within the low-priority window extents (step 812). If at step 812 a low-priority unit of work is present, the operation proceeds to step 806. If at step 812 no unit of work with a priority below the priority threshold is present in the priority queue, the dispatcher searches the appointment queue for a unit of work considered future, e.g. within the future window extents (step 814). If at step 814 one or more units of work are considered future, the dispatcher selects the unit of work closest to the starting extent of the future window and the operation proceeds to step 806.

If at step 814 no unit of work considered future is present in the appointment queue, the dispatcher searches the appointment queue for a unit of work considered past, e.g. within the past extents (step 816). If at step 816 one or more units of work are considered past, the dispatcher selects the unit of work closest to the starting extent of the past window and the operation proceeds to step 806. If at step 816 no unit of work considered past is present in the appointment queue, the dispatcher returns a signal that no unit of work is available to the analytics processor (step 818), with the operation ending thereafter.

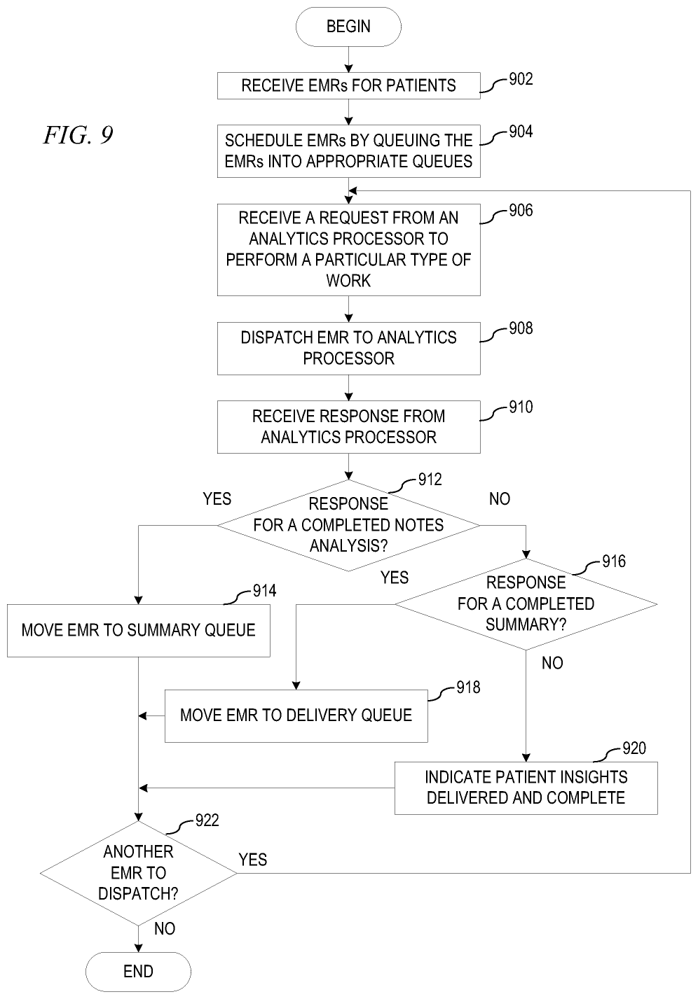

FIG. 9 depicts one example of a flowchart for the operation performed by the anticipatory analytics processing mechanism in queuing and dispatching EMRs for processing in accordance with an illustrative embodiment. As the operation begins, the anticipatory analytics processing mechanism receives a set of EMRs for a set of patients, the set of EMRs being for patients with scheduled appointments as well as patients that walk-in for same-day appointments and those previously seen patients for which lab tests and other results become available (priority patients) (step 902). The anticipatory analytics processing mechanism then schedules the EMRs for processing by queueing the EMRs into appropriate queues as described in FIG. 7 (step 904). The anticipatory analytics processing mechanism receives a request from one or more analytics processor to perform a particular type of work (step 906). The anticipatory analytics processing mechanism dispatches an EMR to a particular analytics processor as described in FIG. 8 (step 908).

Responsive to the analytics processor completing the specific operations, i.e. Notes analysis, Summary, or Delivery, the anticipatory analytics processing mechanism receives a response from the analytics processor (step 910). The anticipatory analytics processing mechanism then determines if the response is for a completed Notes analysis of all Notes in the EMR (step 912). If at step 912 the response is for a completed Notes analysis of all Notes in the EMR, the anticipatory analytics processing mechanism queues the EMR in an appropriate Summary queue, i.e. a priority Summary queue or an appointment Summary queue depending on whether or not the EMR has an associated priority indicator (step 914). If at step 912 the response is not for a completed Notes analysis, the anticipatory analytics processing mechanism determines if the response is for a completed Summary (step 916).

If at step 916 the response is for a completed Summary, the anticipatory analytics processing mechanism queues the EMR in an appropriate Delivery queue, i.e. a priority Delivery queue or an appointment Delivery queue depending on whether or not the EMR has an associated priority indicator (step 918). If at step 916 the response is not for a completed Summary analysis, the anticipatory analytics processing mechanism indicates that the patient insight has been delivered and complete (step 920). From steps 914, 918, or step 920, the anticipatory analytics processing mechanism then determines whether there is another EMR to dispatch (step 922). If at step 922 there is another EMR to dispatch, the operation returns to step 906. If at step 922 there is not another EMR to dispatch, the operation terminates.

The flowcharts and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Thus, the illustrative embodiments provide mechanisms for facilitating patient EMR analytics processing in accordance with patient visits while consuming minimal computing resources. When computing resources are limited, the mechanisms process EMRs for a select group of patients, those in close proximity and prior to scheduled interactions with a medical professional. In one embodiment, the mechanisms classify work into a set of categories, such as priorities categories, appointments categories, or the like. Within the priority categories, the mechanisms order or rank EMRs from highest to lowest priority and, in the case of ties in priority, by order of EMR arrival. Similarly, within appointment categories, the mechanisms order or rank EMRs from an oldest scheduled appointment time (farthest in the past) to a newest scheduled appointment time (farthest in the future), with ties broken by arrival order. The priorities categories may be further classified into a set of subcategories, such as high-priority, low-priority, or the like, and the appointments categories may be further classified into a set of subcategories, such as past appointment, just-missed appointment, imminent appointment, future appointment, or the like. The mechanisms schedule an analysis of corresponding EMRs according to these sets of categories.

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system, for comprising at least one processor and at least one memory, wherein the at least one memory comprises instructions that are executed by the at least one processor to configure the at least one processor to implement an anticipatory analytics processing mechanism for processing electronic medical records (EMRs) of a set of patients using anticipatory analytics to provide patient insights, the method comprising:
responsive to receiving a set of EMRs for a set of patients, queueing each EMR in the set of EMRs into one of a plurality of queues based on a set of work types;
responsive to receiving a request for work by a processing service indicating a work type to be provided, identifying an EMR from the plurality of queues based on the work type to be provided and a predetermined order of work;
dispatching the identified EMR to the processing service; and
responsive to receiving a completion indication from the processing service, advancing the EMR to a queue associated with a next work type category or indicating that patient insight for a patient associated with the EMR is complete.

2. The method of claim 1, where in the set of work types comprises a Notes type, a Summary type, and a Delivery type.

3. The method of claim 1, wherein dispatching the identified EMR to the processing service comprises:
dynamically calculating queue search window extents for past appointment, just-missed appointment, imminent appointment, or future appointment based on the current time-of-day with respect to a request for work from a processing service to facilitate selection of an EMR.

4. The method of claim 1, wherein the predetermined order of work comprises, in order, high-priority EMRs, imminent EMRs, just-missed EMRs, low-priority EMRs, future EMRs, and past EMRs.

5. The method of claim 1, wherein the completion indication is completion of a Notes analysis and wherein, response to the completion indication being the completion of the Notes analysis, the EMR is advanced to a Summary type queue.

6. The method of claim 1, wherein the completion indication is completion of a Summary and wherein, response to the completion indication being the completion of the Summary, the EMR is advanced to a Delivery type queue.

7. The method of claim 1, wherein the completion indication is completion of a Delivery of patient insights and wherein, responsive to the completion indication being the completion of the Delivery of the patient insight, marking the EMR as having patient insights completed.

8. A computer program product comprising a computer readable storage medium having a computer readable program stored therein, wherein the computer readable program, when executed on a data processing system, causes the data processing system to implement an anticipatory analytics processing mechanism for processing electronic medical records (EMRs) of a set of patients using anticipatory analytics to provide patient insights, and further causes the data processing system to:
responsive to receiving a set of EMRs for a set of patients, queue each EMR in the set of EMRs into one of a plurality of queues based on a set of work types;
responsive to receiving a request for work by a processing service indicating a work type to be provided, identify an EMR from the plurality of queues based on the work type to be provided and a predetermined order of work;
dispatch the identified EMR to the processing service; and
responsive to receiving a completion indication from the processing service, advance the EMR to a queue associated with a next work type category or indicating that patient insight for a patient associated with the EMR is complete.

9. The computer program product of claim 8, where in the set of work types comprises a Notes type, a Summary type, and a Delivery type.

10. The computer program product of claim 1, wherein the computer readable program to dispatch the identified EMR to the processing service further causes the data processing system to:
dynamically calculate queue search window extents for past appointment, just-missed appointment, imminent appointment, or future appointment based on the current time-of-day with respect to a request for work from a processing service to facilitate selection of an EMR.

11. The computer program product of claim 8, wherein the predetermined order of work comprises, in order, high-priority EMRs, imminent EMRs, just-missed EMRs, low-priority EMRs, future EMRs, and past EMRs.

12. The computer program product of claim 8, wherein the completion indication is completion of a Notes analysis and wherein, response to the completion indication being the completion of the Notes analysis, the EMR is advanced to a Summary type queue.

13. The computer program product of claim 8, wherein the completion indication is completion of a Summary and wherein, response to the completion indication being the completion of the Summary, the EMR is advanced to a Delivery type queue.

14. The computer program product of claim 8, wherein the completion indication is completion of a Delivery of patient insights and wherein, responsive to the completion indication being the completion of the Delivery of the patient insight, mark the EMR as having patient insights completed.

15. An apparatus comprising:
at least one processor; and
at least one memory coupled to the at least one processor, wherein the at least one memory comprises instructions which, when executed by the at least one processor, cause the at least one processor to implement an anticipatory analytics processing mechanism for processing electronic medical records (EMRs) of a set of patients using anticipatory analytics to provide patient insights, and further cause the at least one processor to:
responsive to receiving a set of EMRs for a set of patients, queue each EMR in the set of EMRs into one of a plurality of queues based on a set of work types;
responsive to receiving a request for work by a processing service indicating a work type to be provided, identify an EMR from the plurality of queues based on the work type to be provided and a predetermined order of work;
dispatch the identified EMR to the processing service; and
responsive to receiving a completion indication from the processing service, advance the EMR to a queue associated with a next work type category or indicating that patient insight for a patient associated with the EMR is complete.

16. The apparatus of claim 15, wherein the instructions to dispatch the identified EMR to the processing service further cause at least one processor to:
dynamically calculate queue search window extents for past appointment, just-missed appointment, imminent appointment, or future appointment based on the current time-of-day with respect to a request for work from a processing service to facilitate selection of an EMR.

17. The apparatus of claim 15, wherein the predetermined order of work comprises, in order, high-priority EMRs, imminent EMRs, just-missed EMRs, low-priority EMRs, future EMRs, and past EMRs.

18. The apparatus of claim 15, wherein the completion indication is completion of a Notes analysis and wherein, response to the completion indication being the completion of the Notes analysis, the EMR is advanced to a Summary type queue.

19. The apparatus of claim 15, wherein the completion indication is completion of a Summary and wherein, response to the completion indication being the completion of the Summary, the EMR is advanced to a Delivery type queue.

20. The apparatus of claim 15, wherein the completion indication is completion of a Delivery of patient insights and wherein, responsive to the completion indication being the completion of the Delivery of the patient insight, mark the EMR as having patient insights completed.
